(19) United States
(12) Patent Application Publication
(10) Pub. No.: US 2011/0112144 A1
(43) Pub. Date: May 12, 2011

(54) METHODS OF PREPARING SUBSTITUTED HETEROCYCLES

(75) Inventors: Matthew Ball, Macclesfield (GB); Martin Francis Jones, Macclesfield (GB); Fiona Ruth Kenley, Macclesfield (GB); David John Pittam, Macclesfield (GB)

(73) Assignee: ASTRAZENECA AB, Södertälje (SE)

(21) Appl. No.: 12/989,860

(22) PCT Filed: Apr. 27, 2009
(86) PCT No.: PCT/GB2009/050424

§ 371 Date: Jan. 6, 2011

Related U.S. Application Data
(62) Division of application No. 61/048,329, filed on Apr. 28, 2008.

(51) Int. Cl. A61K 31/4535; C07D 409/12; A61P 35/00

(52) U.S. Cl. 514/326; 546/213

(57) ABSTRACT

The present disclosure relates to methods of preparing substituted thiophenes, which are useful for the treatment and prevention of cancers. Also disclosed are substituted thiophenes made by the methods disclosed herein.

The present disclosure relates to methods of preparing substituted thiophenes, which are useful for the treatment and prevention of cancers. Also disclosed are substituted thiophenes made by the methods disclosed herein.

Chemotherapy and radiation exposure are currently the major options for the treatment of cancer, but the utility of both these approaches is severely limited by drastic adverse effects on normal tissue, and the frequent development of tumor cell resistance. It is therefore highly desirable to improve the efficacy of such treatments in a way that does not increase the toxicity associated with them. One way to achieve this is by the use of specific sensitizing agents such as those described herein.

An individual cell replicates by making an exact copy of its chromosomes, and then segregating these into separate cells. This cycle of DNA replication, chromosome separation and division is regulated by mechanisms within the cell that maintain the order of the steps and ensure that each step is precisely carried out. Key to these processes are the cell cycle checkpoints (Hartwell et al., Science, Nov. 3, 1989, 246(4930):629-34) where cells may arrest to ensure DNA repair mechanisms have time to operate prior to continuing through the cycle into mitosis. There are two such checkpoints in the cell cycle—the G1/S checkpoint that is regulated by p53 and the G2/M checkpoint that is monitored by the Ser/Thr kinase checkpoint kinase 1 (CHK1).

As the cell cycle arrest induced by these checkpoints is a crucial mechanism by which cells can overcome the damage resulting from radio- or chemotherapy, their abrogation should increase the sensitivity of tumor cells to DNA damaging therapies. Additionally, the tumor specific abrogation of the G1/S checkpoint by p53 mutations in the majority of tumors can be exploited to provide tumor selective agents. One approach to the design of chemosensitizers that abrogate the G2/M checkpoint is to develop inhibitors of the key G2/M regulatory kinase CHK1, and this approach has been shown to work in a number of proof-of-concept studies. (Koniaras et al., Oncogene, 2001, 20:7453; Luo et al., Neoplasia, 2001, 3:411; Busby et al., Cancer Res., 2000, 60:2108; Jackson et al., Cancer Res., 2000, 60:566).

The substituted thiophenes of the present invention have been shown to be potent inhibitors of the CHK1 kinase (WO 2005/066163). By inhibiting CHK1, the presently disclosed substituted heterocycles possess the ability to prevent cell cycle arrest at the G2/M checkpoint in response to DNA damage. These compounds are accordingly useful for their anti-proliferative (such as anti-cancer) activity and are therefore useful in methods of treatment of the human or animal body. Such methods include treatment of disease states associated with cell cycle arrest and cell proliferation such as cancers (solid tumors and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Current methods to access these substituted thiophenes have several disadvantages, which cause them to be nearly impractical for scale-up preparations. Difficulties have been encountered with a bromination reaction, and an amide bond formation that requires a large excess of one of the starting materials and a relatively large amount of AlMe₃. This latter reagent is pyrophoric and environmentally unfriendly. Purification of intermediates in currently known methods can be operationally laborious, given the multiple chromatographies, filtrations and solvent exchanges that are required.

Accordingly, better methods of synthesizing these valuable compounds are needed. The present invention provides methods of preparing substituted thiophenes that use no metal-catalyzed couplings or brominations, thus obviating the need for chromatography, which can effectively limit the scale at which a reaction is run. Recrystallization procedures have replaced the solvent exchange, which minimizes degradation of the final product. Overall yield has increased such that far less starting materials are required.

One embodiment of the invention provides a method of preparing a compound of formula I:

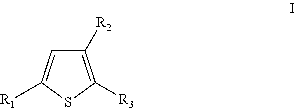

or a pharmaceutically acceptable salt thereof,

wherein

R₁ is an aryl ring optionally substituted with one or more R₄ groups selected from halogen, C₁₋₆alkoxy, C₁₋₆alkoxycarbonyl, C₁₋₆alkyl, C₂₋₆alkenyl, C₂₋₆alkynyl, amido, amino, aryl, aryloxy, carboxy, cycloalkyl, heterocyclyl, and hydroxy;

R₂ is —NHC(O)NHR₅, where R₅ is selected from H, C₁₋₆alkyl, C₁₋₆alkoxycarbonyl, aryl, cycloalkyl, and heterocyclyl;

R₃ is —C(O)NR₆R₇, where R₆ and R₇ are each independently selected from H, C₁₋₆alkyl, cycloalkyl and a 5, 6, or 7-membered heterocyclyl ring containing at least one nitrogen atom, provided R₆ and R₇ are not both H;

comprising

(a) reacting a 2-thioacetamide compound with a compound of formula II

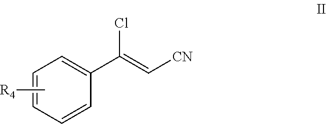

to produce a thiophene intermediate; and

(b) further reacting the thiophene intermediate to form the compound of formula I.

An “intermediate” as used herein refers to a compound that is formed as an intermediate product between the starting material and the final compound of formula I. “Reaction mixture” as used herein refers to a solution or slurry comprising at least one product of a chemical reaction between reagents, as well as by-products, e.g., impurities (including compounds with undesired stereochemistries), solvents, and any remaining reagents, such as starting materials. In one embodiment, the reaction mixture is a slurry, where a slurry can be a composition comprising at least one solid and at least one liquid (such as water, acid, or a solvent), e.g., a suspension or a dispersion of solids. In one embodiment, an intermediate is not isolated from the reaction mixture prior to carrying out the next transformation.

In one embodiment, a reaction step can be performed in a large scale. In one embodiment, “large scale” refers to the use of at least 1 gram of a starting material, intermediate or reagent, such as the use of at least 2 grams, at least 5 grams, at least 10 grams, at least 25 grams, at least 50 grams, at least 100 grams, at least 500 g, at least 1 kg, at least 5 kg, at least 10 kg, at least 25 kg, at least 50 kg, or at least 100 kg.

In one embodiment, the 2-thioacetamide compound has the following formula III:

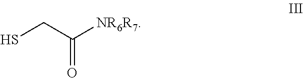

In one embodiment, the 2-thioacetamide compound can be present in a reaction mixture slurry, which is reacted with the compound of formula II. In one embodiment, the reaction of the 2-thioacetamide compound with the compound of formula II can take place in the presence of a nucleophilic base. In another embodiment, the base can serve to form the 2-thioacetamide compound in situ by deacetylating a precursor thioacetyl intermediate. In a further embodiment, the base can be selected from sodium methoxide, sodium hydroxide, sodium or potassium ethoxide, sodium or potassium t-butoxide, and sodium t-amylate. In a further embodiment, the base can be sodium methoxide. The base may be added before or after the compound of formula II. The base may be present, for example, in about 1.1-3.5 equivalents, such as about 1.5 equivalents. The compound of formula II may be present in, for example, about 0.9 equivalents. The reaction can take place in any solvent deemed suitable by one of ordinary skill in the art. In one embodiment, the solvent can be 2-methyltetrahydrofuran.

The reaction can be carried out at about 0-40° C. In one embodiment, the method further comprises purifying the resulting thiophene intermediate by crystallization. In a further embodiment, the crystallization can be performed at about 0-5° C. from 1-3 days.

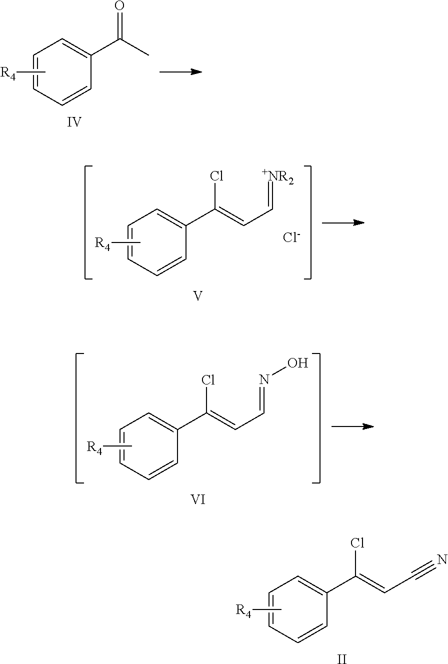

The compound of formula II can be formed by treating acetophenone IV with a Vilsmeier reagent to give iminium species V. Variable R on iminium species V can be an alkyl group, such as a methyl group. The acetophenone can be added either before or after the formation of the Vilsmeier reagent. Suitable Vilsmeier reagents can be prepared from DMF and POCl₃, DMF and oxalyl chloride, DMF and PCl₅, DMF and thionyl chloride, and DMF, POCl₃, and PCl₅. In one embodiment, DMF and POCl₃ can be used. While DMF can be the bulk solvent, in a further embodiment, about 2 equivalents of DMF in toluene or acetonitrile can be used. In another embodiment, instead of DMF, a different dialkyl formamide HC(O)NR₂ can be used, including formamides where the R groups together form a cycle such as cycloalkyls and morpholine. Alternatives to the Cl⁻ counterion of iminium V include perchlorate and PF₆⁻ salts.

The iminium V can be treated with hydroxylamine hydrochloride, phosphate or sulfate to form an oxime VI, which further reacts to provide the compound of formula II. The hydroxylamine salt and iminium V can be added in either order. In one embodiment, the oxime VI can be isolated prior to conversion to the compound of formula II. In another embodiment, oxime VI can react in situ to yield the compound of formula II. In one embodiment, purification of the compound of formula II by crystallization can be carried out on the same day as its formation.

Another embodiment of the invention provides a method of preparing a compound of formula I:

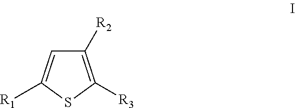

or a pharmaceutically acceptable salt thereof,

wherein

R₁ is an aryl ring optionally substituted with one or more R₄ groups selected from halogen, C₁₋₆alkoxy, C₁₋₆alkoxycarbonyl, C₁₋₆alkyl, C₂₋₆alkenyl, C₂₋₆alkynyl, amido, amino, aryl, aryloxy, carboxy, cycloalkyl, heterocyclyl, and hydroxy;

R₂ is —NHC(O)NHR₅, where R₅ is selected from H, C₁₋₆alkyl, C₁₋₆alkoxycarbonyl, aryl, cycloalkyl, and heterocyclyl;

R₃ is —C(O)NR₆R₇, where R₆ and R₇ are each independently selected from H, C₁₋₆alkyl, cycloalkyl and a 5, 6, or 7-membered heterocyclyl ring containing at least one nitrogen atom, provided R₆ and R₇ are not both H;

comprising

(a) reacting HNR₆R₇ with a haloacetyl halide to form a haloacetamide intermediate;

(b) reacting the haloacetamide intermediate with a thioacetic acid salt to form a thioacetyl intermediate;

(c) deacetylating the thioacetyl intermediate to form a 2-thioacetamide intermediate;

(d) reacting the 2-thioacetamide intermediate with a compound of formula II

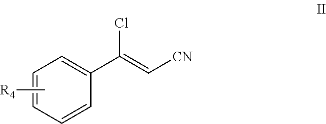

to form a thiophene intermediate; and

(e) further reacting the thiophene intermediate to form the compound of formula I.

In one embodiment, a molar excess of haloacetyl halide is added to HNR₆R₇, such as about 1.5 equivalents. In one embodiment, the haloacetyl halide can be chloroacetyl chloride or chloroacetyl bromide. In another embodiment, a base can be added with the haloacetyl halide, such as pyridine, diisopropylamine, triethylamine, 2,6-lutidine, and N,N-dimethylaminopyridine. In a further embodiment, the base can be pyridine. The base may be added in molar excess of the HNR₆R₇, such as 1.2 equivalents.

In one embodiment, the haloacetamide intermediate is not isolated prior to addition of the thioacetic acid salt. In another embodiment, the haloacetamide intermediate is isolated prior to treatment with the thioacetic acid salt. In one embodiment, the haloacetamide intermediate can be ClCH₂C(O)NR₆R₇. In one embodiment, the thioacetic acid salt can be an alkaline earth salt, such as potassium thioacetate or tetramethylammonium thioacetate. The thioacetic acid salt can be added in molar excess of the haloacetamide intermediate, such as about 1.5 equivalents. The reactions can take place in any solvent deemed suitable by one of ordinary skill in the art. In one embodiment, the addition of thioacetic acid salt can occur in a biphasic water/2-methyltetrahydrofuran system. Anhydrous tetrahydrofuran or anhydrous 2-methyltetrahydrofuran can also be used.

Another embodiment of the invention provides a method of preparing a compound of formula I:

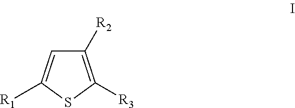

or a pharmaceutically acceptable salt thereof,

wherein

R₁ is an aryl ring optionally substituted with one or more R₄ groups selected from halogen, C₁₋₆alkoxy, C₁₋₆alkoxycarbonyl, C₁₋₆alkyl, C₂₋₆alkenyl, C₂₋₆alkynyl, amido, amino, aryl, aryloxy, carboxy, cycloalkyl, heterocyclyl, and hydroxy;

R₂ is —NHC(O)NHR₅, where R₅ is selected from H, C₁₋₆alkyl, C₁₋₆alkoxycarbonyl, aryl, cycloalkyl, and heterocyclyl;

R₃ is —C(O)NR₆R₇, where R₆ and R₇ are each independently selected from H, C₁₋₆alkyl, cycloalkyl and a 5, 6, or 7-membered heterocyclyl ring containing at least one nitrogen atom, provided R₆ and R₇ are not both H;

comprising

(a) reacting a thiophene intermediate of formula VII, or a pharmaceutically acceptable salt thereof.

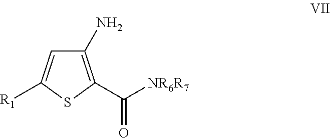

with an isocyanate to form a ureido intermediate;

(b) reacting the ureido intermediate with a base to form a urea intermediate; and

(c) further reacting the urea intermediate to form the compound of formula I.

In one embodiment, the ureido intermediate is a compound of formula VIII

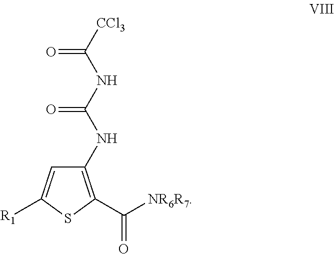

In one embodiment, a molar excess of isocyanate is added to the intermediate of formula IV, such as about up to about 2 equivalents. In a further embodiment, the isocyanate can be trichloroacetyl isocyanate. In another embodiment, the solvent can be selected from tetrahydrofuran, acetonitrile and methyl tert-butyl ether, such as tetrahydrofuran.

In one embodiment, the ureido intermediate can be isolated prior to reacting with a base. In another embodiment, the ureido intermediate can be in a reaction mixture slurry when the base is added. In one embodiment, the base can be added in molar excess to the ureido intermediate, such as about 2.5 equivalents. The base may be selected from triethylamine, diisopropylethylamine, methylamine, and ethanol magnesium salt and methanol. In one embodiment, the base can be triethylamine.

In one embodiment, the reaction can be performed for about 2.5 to about 4 hours. The reactions can take place in any solvent deemed suitable by one of ordinary skill in the art. In one embodiment, the solvent can be chosen from tetrahydrofuran, acetonitrile, dichloromethane, toluene, benzene, diethyl ether, dioxane, hexane, and carbon tetrachloride. In a further embodiment, the solvent can be tetrahydrofuran. In one embodiment, the resulting urea intermediate can be purified by crystallization through portionwise addition of water.

In an alternative embodiment, formation of the compound of formula I comprises

(a) reacting a thiophene intermediate of formula VII, or a pharmaceutically acceptable salt thereof,

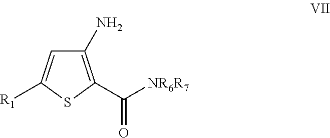

with one or more reagents to form a urea intermediate; and

(b) further reacting the urea intermediate to form the compound of formula I.

In one embodiment, the one or more reagents may be selected from trimethylsilyl isocyanate followed by acidic workup; sodium, potassium, or silver cyanate; isocyanic acid; monochloroacetyl isocyanate followed by NaOMe; carbodiimide followed by urea; urea in refluxing pyridine; nitrourea; benzyl isocyanate followed by NaOH; benzyloxyisocyanate followed by hydrogenolysis; phosgene, ammonia, and benzene; thiourea, triethylamine, and methanol; chlorocarbonyl isocyanate followed by ammonia; ethyl chloroformate followed by ammonia; and silicon tetraisocyanate.

In one embodiment, the ureido intermediate bears an acid-labile protecting group such that reacting it with a base provides a protected urea intermediate. This intermediate can then be treated with acid to remove the acid-labile protecting group and obtain the compound of formula I. In one embodiment, the protected urea intermediate can be isolated prior to reacting with acid. In another embodiment, the acid can be added to a reaction mixture slurry that comprises the protected urea intermediate. The acid may be added in molar excess to the protected urea intermediate, such as about 3 equivalents. In one embodiment, the protected urea intermediate can bear a carbamate protecting group, such as a t-butylcarbamate protecting group. Other suitable carbamate protecting groups include, for example, 2,2,2-trichloroethyl carbamate, 2-trimethylsilylethyl carbamate, allyl carbamate, benzyl carbamate, 2-phenylethyl carbamate, and 2-chloroethyl carbamate. In addition, other useful protecting groups include, for example, formamide, benzamide, acetamide, pent-4-enamide, o-nitrophenylacetamide, o-nitrophenoxyacetamide, allyl, N-4-methoxybenzylamine, and diphenylphosphinamide.

A variety of acidic conditions may be used to effect transformation of a protected intermediate to a compound of formula I. These include anhydrous or aqueous HCl in methanol, ethanol, tetrahydrofuran, or ethyl acetate; acetyl chloride in methanol; trifluoroacetic acid with or without a sulfide; toluene sulfonic acid; sulfuric acid in dioxane; bromocatechol borane; trimethylsilyl chloride in phenol/dichloromethane; tetrachlorosilane in phenol/dichloromethane; trimethylsilyl triflate with a sulfide; tert-butyldimethylsilyl triflate; methane sulfonic acid in dioxane/dichloromethane; silica gel; ceric ammonium nitrate in acetonitrile; and zinc in tetrahydrofuran. In a further embodiment, the acid can be aqueous HCl in methanol. Other conditions to remove acid-labile protecting groups include palladium catalyzed reductions, H₂ with a catalyst, samarium iodide, and iodine in tetrahydrofuran. Following removal of the acid labile protecting group, a base can be added, such as triethylamine or sodium carbonate.

The compound of formula I may be further purified by filtering a warm, such as about 30° C., suspension of the compound through a glass filter, then cooling to about 10-15° C., adding water and inducing crystallization with a seed crystal of the compound of formula I. Further addition of water with stirring can complete the crystallization process.

Another embodiment of the invention provides a method of preparing a compound of formula I

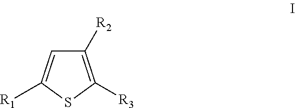

or a pharmaceutically acceptable salt thereof,

wherein

R₁ is an aryl ring optionally substituted with one or more R₄ groups selected from halogen, C₁₋₆alkoxy, C₁₋₆alkoxycarbonyl, C₁₋₆alkyl, C₂₋₆alkenyl, C₂₋₆alkynyl, amido, amino, aryl, aryloxy, carboxy, cycloalkyl, heterocyclyl, and hydroxy;

R₂ is —NHC(O)NHR₅, where R₅ is selected from H, C₁₋₆alkyl, C₁₋₆alkoxycarbonyl, aryl, cycloalkyl, and heterocyclyl;

R₃ is —C(O)NR₆R₇, where R₆ and R₇ are each independently selected from H, C₁₋₆alkyl, cycloalkyl and a 5, 6, or 7-membered heterocyclyl ring containing at least one nitrogen atom, provided R₆ and R₇ are not both H;

comprising

(a) reacting HNR₆R₇ with a haloacetyl halide to form a haloacetamide intermediate;

(b) reacting the haloacetamide intermediate with a thioacetic acid salt to form a thioacetyl intermediate;

(c) deacetylating the thioacetyl intermediate to form a 2-thioacetamide intermediate;

(d) reacting the 2-thioacetamide intermediate with a compound of formula II

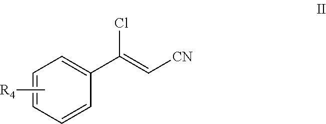

to form a thiophene intermediate of formula VII

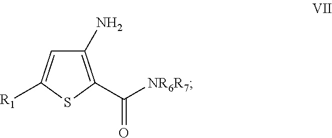

(e) reacting the thiophene intermediate of formula VII with an isocyanate to form a ureido intermediate;

(f) reacting the ureido intermediate with a base to form a protected intermediate; and

(g) reacting the protected intermediate with an acid to form the compound of formula I. Another embodiment of the invention provides a method of preparing a compound of formula I

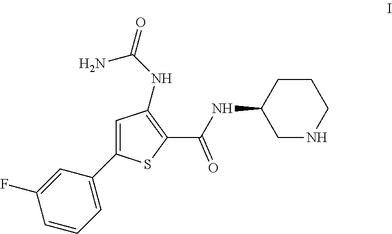

or a pharmaceutically acceptable salt thereof,

comprising the following steps:

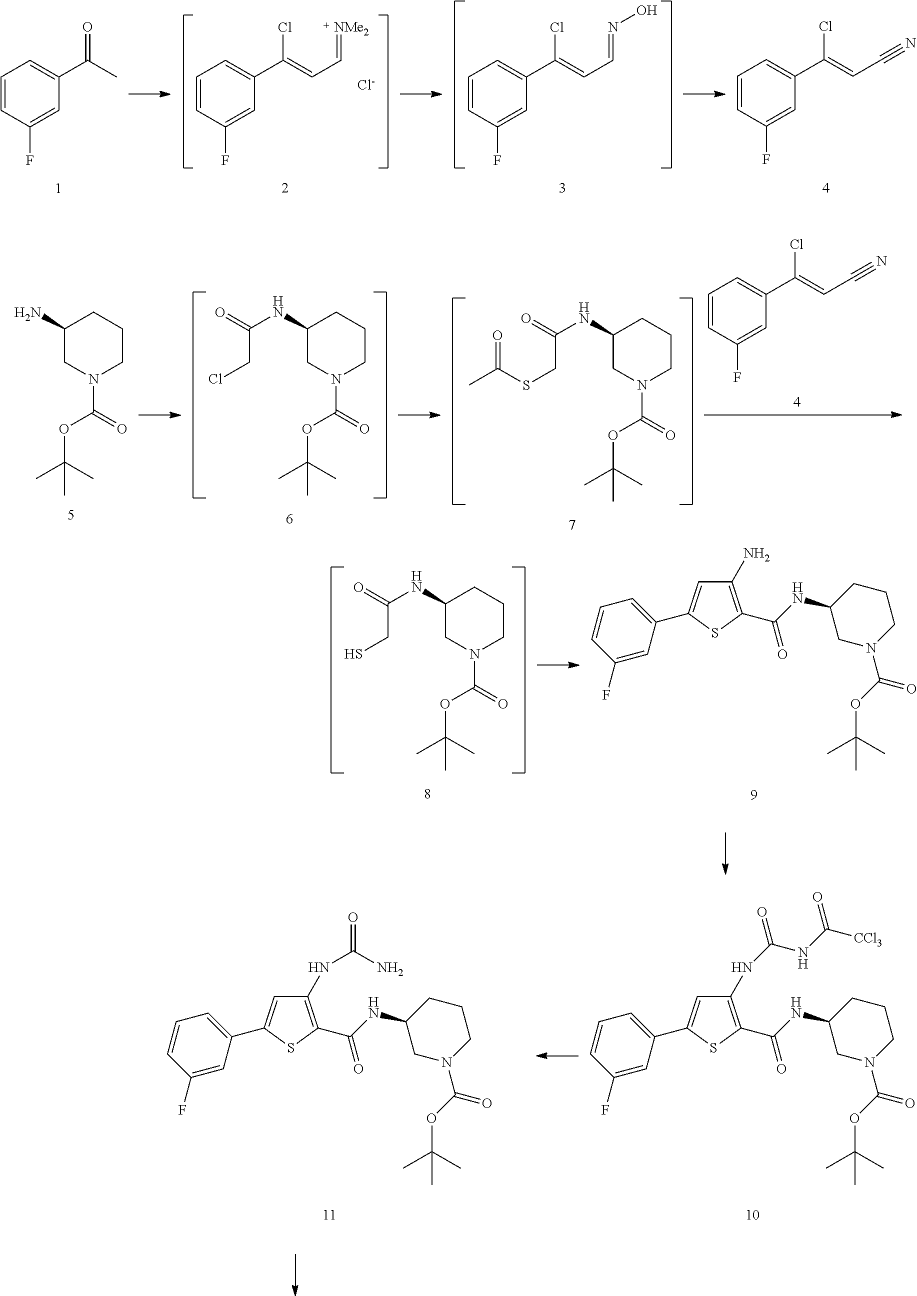

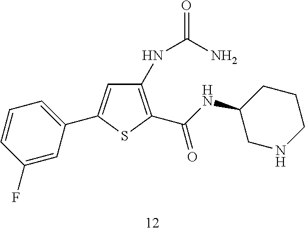

and optionally, further reacting compound 12 to form a pharmaceutically acceptable salt thereof.

Brackets indicate intermediates that are not isolated prior to further reaction. Compound 1 can be treated with POCl₃ in DMF, followed by addition of hydroxylamine hydrochloride to give compound 4. Compound 5 can be reacted with chloroacetyl chloride and pyridine to provide intermediate 6, which gives intermediate 7 upon treatment with potassium thioacetate. Addition of compound 4 and sodium methoxide to intermediate 7 results in formation of compound 9. Reaction of compound 9 with trichloroacetyl isocyanate can give compound 10, which can be transformed to compound II upon treatment with alcoholic triethylamine. Compound II can be reacted with methanolic HCl to provide compound 12. Salts of compound 12 can be formed by methods described herein below or by methods well known in the art.

It will be clear to one of skill in the art that the preceding process can be used to make other compounds of formula I or pharmaceutically acceptable salts thereof using the appropriate starting materials which may be commercially available or can be made by analogous methods described herein or by methods known in the art.

One embodiment provides a compound of formula I

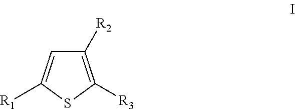

or a pharmaceutically acceptable salt thereof,

wherein

R₁ is an aryl ring optionally substituted with one or more R₄ groups selected from halogen, C₁₋₆alkoxy, C₁₋₆alkoxycarbonyl, C₁₋₆alkyl, C₂₋₆alkenyl, C₂₋₆alkynyl, amido, amino, aryl, aryloxy, carboxy, cycloalkyl, heterocyclyl, and hydroxy;

R₂ is —NHC(O)NHR₅, where R₅ is selected from H, C₁₋₆alkyl, C₁₋₆alkoxycarbonyl, aryl, cycloalkyl, and heterocyclyl;

R₃ is —C(O)NR₆R₇, where R₆ and R₇ are each independently selected from H, C₁₋₆alkyl, cycloalkyl and a 5, 6, or 7-membered heterocyclyl ring containing at least one nitrogen atom, provided R₆ and R₇ are not both H;

made by any of the processes disclosed herein. Another embodiment provides a composition comprising a compound of formula I made by any of the processes disclosed herein and a pharmaceutically acceptable carrier.

The following substituents for the variable groups contained in formulae I-VIII are further embodiments of the invention. Such specific substituents may be used, where appropriate, with any of the definitions, claims or embodiments defined hereinbefore or hereinafter.

In one embodiment, R₄ is halogen, such as fluoro. In another embodiment, R₁ is an aryl ring mono-substituted with a fluoro group. In another embodiment, R₅ is H. In another embodiment, R₅ is C₁₋₆alkoxycarbonyl.

In one embodiment, R₆ is a 5, 6, or 7-membered heterocyclyl ring and R₇ is H. In another embodiment, R₆ is a 6-membered saturated heterocyclyl containing one nitrogen atom. In a further embodiment, the nitrogen atom is protected by a carbamate protecting group, such as a t-butoxycarbonyl group.

It is to be understood that all embodiments are exemplary and explanatory only and are not restrictive of the invention as claimed.

It should be noted that, as used in this specification and the appended claims, the singular forms “a,” “an,” and “the” include plural referents unless the content clearly dictates otherwise. Thus, for example, reference to a method containing “a compound” includes a mixture of two or more compounds. It should also be noted that the term “or” is generally employed in its sense including “and/or” unless the content clearly dictates otherwise. Unless otherwise specified, the chemical groups refer to their unsubstituted and substituted forms.

The term “compound” as used herein refers to a neutral compound (e.g. a free base), and salt forms thereof (such as pharmaceutically acceptable salts). The compound can exist in anhydrous form, or as a hydrate, or as a solvate. The compound may be present as stereoisomers (e.g., enantiomers and diastereomers), and can be isolated as enantiomers, racemic mixtures, diastereomers, and mixtures thereof. The compound in solid form can exist in various crystalline and amorphous forms.

The term “Cₘ₋ₙ” or “Cₘ₋ₙ group” used alone or as a prefix, refers to any group having m to n carbon atoms.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂₋C₁₂alkenyl, C₂₋C₁₀alkenyl, and C₂₋C₆alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkoxy” as used herein refers to an alkyl group attached to an oxygen (—O-alkyl-). Exemplary alkoxy groups include, but are not limited to, groups with an alkyl, alkenyl or alkynyl group of 1-12, 1-8, or 1-6 carbon atoms, referred to herein as C₁-C₁₂alkoxy, C₁-C₈alkoxy, and C₁-C₆alkoxy, respectively. Exemplary alkoxy groups include, but are not limited to methoxy, ethoxy, etc. Similarly, exemplary “alkenoxy” groups include, but are not limited to vinyloxy, allyloxy, butenoxy, etc.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

Alkyl groups can optionally be substituted with or interrupted by at least one group selected from alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl.

The term “alkynyl” as used herein refers to an unsaturated straight or branched hydrocarbon having at least one carbon-carbon triple bond, such as a straight or branched group of 2-12, 2-8, or 2-6 carbon atoms, referred to herein as C₂-C₁₂alkynyl, C₂₋C₈alkynyl, and C₂₋C₆alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl, etc.

The term “amide” or “amido” as used herein refers to a radical of the form —RₐC(O)N(R_{b})—, —RₐC(O)N(R_{b})R_{c}—, or —C(O)NR_{b}R_{c}, wherein R_{b} and R_{c} are each independently selected from alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, and nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring. The term “carboxamido” refers to the structure —C(O)NR_{b}R_{c}.

The term “amine” or “amino” as used herein refers to a radical of the form —NR_{d}Rₑ, —N(R_{d})Rₑ—, or —RₑN(R_{d})R_{f}— where R_{d}, Rₑ, and R_{f} are independently selected from alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, and nitro. The amino can be attached to the parent molecular group through the nitrogen, R_{d}, Rₑ or R_{f}. The amino also may be cyclic, for example any two of R_{d}, Rₑ or R_{f} may be joined together or with the N to form a 3- to 12-membered ring, e.g., morpholino or piperidinyl. The term amino also includes the corresponding quaternary ammonium salt of any amino group, e.g., —[N(R_{d})(Rₑ)(R_{f})]⁺. Exemplary amino groups include aminoalkyl groups, wherein at least one of R_{d}, Rₑ, or R_{f} is an alkyl group.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl.

The term “arylalkyl” as used herein refers to an aryl group having at least one alkyl substituent, e.g. -aryl-alkyl-. Exemplary arylalkyl groups include, but are not limited to, arylalkyls having a monocyclic aromatic ring system, wherein the ring comprises 6 carbon atoms. For example, “phenylalkyl” includes phenylC₄alkyl, benzyl, 1-phenylethyl, 2-phenylethyl, etc.

The term “carbamate” as used herein refers to a radical of the form —R_{g}OC(O)N(Rₕ)—, —R_{g}OC(O)N(Rₕ)Rᵢ₋, or —OC(O)NRₕRᵢ, wherein R_{g}, Rₕ and Rᵢ are each independently selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonyl, and sulfonamide. Exemplary carbamates include, but are not limited to, arylcarbamates or heteroaryl carbamates, e.g., wherein at least one of R_{g}, Rₕ and Rᵢ are independently selected from aryl or heteroaryl, such as phenyl and pyridinyl.

The term “carbonyl” as used herein refers to the radical —C(O)—.

The term “carboxamido” as used herein refers to the radical —C(O)NRR′, where R and R′ may be the same or different. R and R′ may be selected from, for example, alkyl, aryl, arylalkyl, cycloalkyl, formyl, haloalkyl, heteroaryl and heterocyclyl.

The term “carboxy” as used herein refers to the radical —COOH or its corresponding salts, e.g. —COONa, etc.

The term “cyano” or “nitrile” as used herein refers to the radical —CN.

The term “cycloalkoxy” as used herein refers to a cycloalkyl group attached to an oxygen.

The term “cycloalkyl” as used herein refers to a monovalent saturated or unsaturated cyclic, bicyclic, or bridged bicyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclohexenes, cyclopentanes, cyclopentenes, cyclobutanes and cyclopropanes. Cycloalkyl groups may be substituted with alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl. Cycloalkyl groups can be fused to other cycloalkyl, aryl, or heterocyclyl groups. Fused rings generally refer to at least two rings sharing two atoms therebetween.

The term “ether” refers to a radical having the structure —Rₗ—O—Rₘ—, where Rₗ and Rₘ can independently be alkyl, aryl, cycloalkyl, heterocyclyl, or ether. The ether can be attached to the parent molecular group through Rₗ or Rₘ. Exemplary ethers include, but are not limited to, alkoxyalkyl and alkoxyaryl groups. Ether also includes polyethers, e.g., where one or both of Rₗ and Rₘ are ethers.

The terms “halo” or “halogen” or “Hal” as used herein refer to F, Cl, Br, or I. The term “haloalkyl” as used herein refers to an alkyl group substituted with one or more halogen atoms.

The term “heteroaryl” as used herein refers to a mono-, bi-, or other multi-cyclic, aromatic ring system containing one or more heteroatoms, for example 1 to 4 heteroatoms, such as nitrogen, oxygen, and sulfur. Heteroaryls can be substituted with one or more substituents including alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2 to 5 carbon atoms and 1 to 3 heteroatoms.

The terms “heterocycle,” “heterocyclyl,” or “heterocyclic” as used herein refer to a saturated, partially unsaturated, or unsaturated 4-12 membered ring containing at least one heteroatom independently selected from nitrogen, oxygen, and sulfur. Unless otherwise specified, the heteroatom may be carbon or nitrogen linked, a —CH₂— group can optionally be replaced by a —C(O)—, and a ring sulfur atom may be optionally oxidized to form a sulfinyl or sulfonyl group. Heterocycles can be aromatic (heteroaryls) or non-aromatic. Heterocycles can be substituted with one or more substituents including alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl.

Heterocycles also include bicyclic, tricyclic, and tetracyclic groups in which any of the above heterocyclic rings is fused to one or two rings independently selected from aryls, cycloalkyls, and heterocycles. Exemplary heterocycles include 1H-indazolyl, 2-pyrrolidonyl, 2H, 6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azepanyl, azetidinyl, aziridinyl, azocinyl, benzimidazolyl, benzofuranyl, benzofuryl, benzothiofuranyl, benzothienyl, benzothiophenyl, benzodioxolyl, benzoxazolyl, benzthiophenyl, benzthiazolyl, benzotriazolyl, benzotetrazolyl, benzisoxazolyl, benzthiazole, benzisothiazolyl, benzimidazolyls, benzimidazalonyl, carbazolyl, 4aH-carbazolyl, b-carbolinyl, chromanyl, chromenyl, cinnolinyl, decahydroquinolinyl, dihydroindolyl, dihydropyranyl, dihydrothienyl, dithiazolyl, 2H,6H-1,5,2-dithiazinyl, dioxolanyl, furyl, 2,3-dihydrofuranyl, 2,5-dihydrofuranyl, dihydrofuro[2,3-b]tetrahydrofuranyl, furanyl, furazanyl, homopiperidinyl, imidazolyl, imidazolidinyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isothiazolidinyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxiranyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, piperidinyl, pteridinyl, piperidonyl, 4-piperidonyl, purinyl, pyranyl, pyrrolidinyl, pyrrolinyl, pyrrolidinyl, pyrazinyl, pyrazolyl, pyrazolidinyl, pyrazolinyl, pyrazolyl, pyridazinyl, pyridooxazolyl, pyridoimidazolyl, pyridothiazolyl, pyridinyl, N-oxide-pyridinyl, pyridyl, pyrimidinyl, pyrimidyl, pyrrolidinyl, pyrrolidinyl, pyrrolidin-2-onyl, pyrrolinyl, pyrrolyl, pyridinyl, quinazolinyl, quinolinyl, 4H-quinolizinyl, quinoxalinyl, quinuclidinyl, carbolinyl, tetrahydrofuranyl, tetrahydroisoquinolinyl, tetrahydroisoquinolyl, tetrahydropyranyl, tetrazolyl, thiophanyl, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thiazolidinyl, thianthrenyl, thiazolyl, thienyl, thienothiazolyl, thienooxazolyl, thienoimidazolyl, thiomorpholinyl, thiophenyl, thiopyranyl, thiiranyl, triazinyl, 1,2,3-triazolyl, 1,2,4-triazolyl, 1,2,5-triazolyl, 1,3,4-triazolyl, and xanthenyl.

The terms “hydroxy” and “hydroxyl” as used herein refers to the radical —OH. The term “hydroxyalkyl” as used herein refers to a hydroxy radical attached to an alkyl group.

The term “nitro” as used herein refers to the radical —NO₂. The term “phenyl” as used herein refers to a 6-membered carbocyclic aromatic ring. The phenyl group can also be fused to a cyclohexane or cyclopentane ring. Phenyl can be substituted with one or more substituents including alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl.

The term “sulfonamide” as used herein refers to a radical having the structure —N(Rᵣ)—S(O)₂—R_{S}— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

The term “sulfide” as used herein refers to the radical having the structure R_{z}S—, where R_{z} can be alkoxy, alkyl, alkenyl, alkynyl, amide, amino, aryl, arylalkyl, carbamate, carboxy, cycloalkyl, ester, ether, formyl, haloalkyl, heteroaryl, heterocyclyl, and ketone. The term “alkylsulfide” as used herein refers to an alkyl group attached to a sulfur atom. Exemplary sulfides include “thio,” which as used herein refers to an —SH radical.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. For example, acids having two acidic groups may form salts with a basic compound in the ratio of 1:1 or 1:2 acid:basic compound. In one embodiment, the salt is a fumarate salt. In another embodiment, the salt is a hemi-fumarate salt.

Compounds having an amino moiety may form pharmaceutically acceptable salts with various amino acids, in addition to the acids mentioned above. Compounds that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present invention. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

### EXAMPLES

The compounds of the present invention can be prepared in a number of ways well known to one skilled in the art of organic synthesis. More specifically, compounds of the invention may be prepared using the reactions and techniques described herein. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated.

The starting materials for the examples are either commercially available or are readily prepared by standard methods from known materials. In the following examples, the conditions are as follows, unless stated otherwise:
(i) temperatures are given in degrees Celsius (° C.); operations are carried out at room temperature or ambient temperature, such as a range of about 18-25° C., unless otherwise stated;
(ii) in general, the course of reactions was followed by TLC or liquid chromatography/mass spectroscopy (LC/MS), and reaction times are given for illustration only;
(iii) final products have been analyzed using proton nuclear magnetic resonance (NMR) spectra and/or mass spectra data;
(iv) yields are given for illustration only and are not necessarily those that can be obtained by diligent process development; preparations can be repeated if more material is desired;
(v) when given, nuclear magnetic resonance (NMR) data is in the form of delta (6) values for major diagnostic protons, given in part per million (ppm) relative to tetramethylsilane (TMS) as an internal standard, determined at either 300 or 400 MHz in d₆-DMSO or d₄-MeOD;
(vi) chemical symbols have their usual meanings in the art; and
(vii) solvent ratio is given in volume:volume (v/v) terms.

### Example 1

### Synthesis of (Z)-3-Chloro-3-(3-fluorophenyl)-acrylonitrile from 3′-Fluoroacetophenone

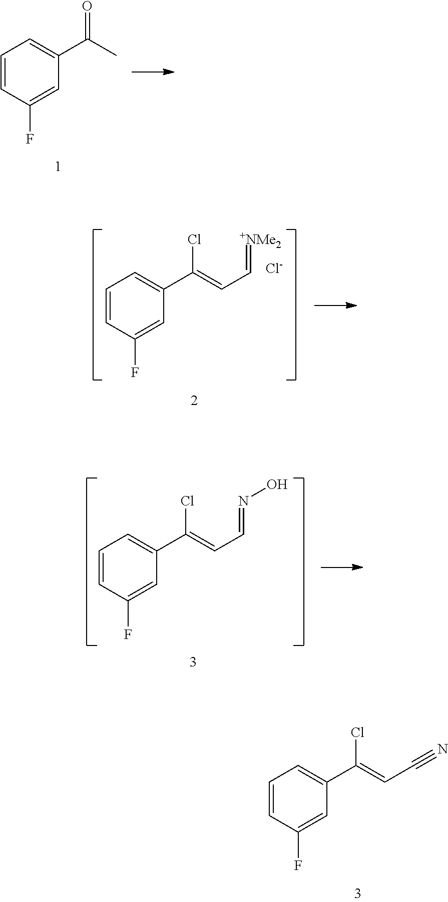

To a solution of 3′-fluoroacetophenone (80.0 g, 0.579 mol) in N,N-dimethyl formamide (560 ml) at about 40° C. was added phosphoryl chloride (92.50 ml, 1.01 mol) dropwise, maintaining the temperature at about 39-41° C. during the addition. The resulting reaction mixture was stirred at about 40° C. overnight before sampling for conversion to 2 by HPLC.

To the resulting reaction mixture was added a solution of hydroxylamine hydrochloride (45.17 g, 0.637 mol) in N,N-dimethyl formamide (240 ml) dropwise, maintaining the temperature at about 39-45° C. during the addition, followed by a line-wash of N,N-dimethyl formamide (40 ml). After stirring at about 40° C. for 15 min, the reaction mixture was sampled for conversion to 4 before cooling to about 15-20° C. and addition of water (800 ml) dropwise, maintaining the temperature between about 17 to about 21° C. The reaction mixture was then cooled to about 5° C. and held at this temperature for a further 20 min before filtration of the solid, displacement washing with two separate portions of water (2×240 ml) and drying at about 40° C. overnight to afford the title compound as a pale yellow solid (74.24 g, 71% yield).

1H NMR (400 MHz, DMSO-d6) δ: 7.72-7.65 (m, 2H), 7.63-7.56 (m, 1H), 7.49-7.42 (m, 1H), 7.03 (s, 1H).

13C NMR (400 MHz, DMSO-d6) δ: 162.0 (d, J=245 Hz), 149.3 (d, J=3 Hz), 135.6 (d, J=8 Hz), 131.1 (d, J=9 Hz), 123.3 (d, J=3 Hz), 118.8 (d, J=21 Hz), 115.8, 113.8 (d, J=24 Hz), 89.3.

### Example 2

### Synthesis of tert-butyl (3S)-3-({[3-amino-5-(3-fluorophenyl)thiophen-2-yl]carbonyl}amino)piperidine-1-carboxylate from (S)-1-Boc-3-aminopiperidine and compound 4

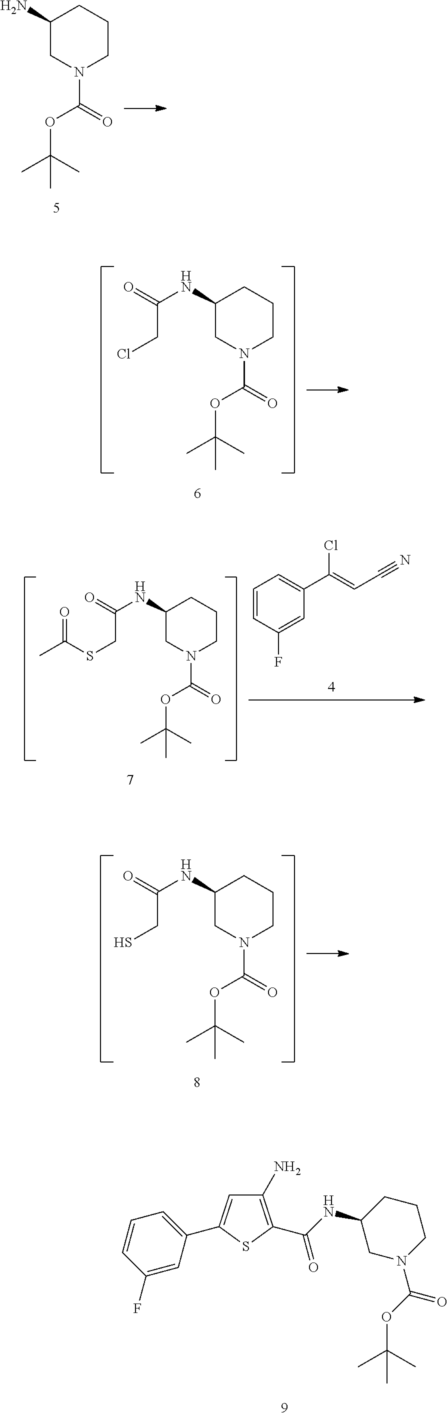

1-Boc-3-(S)-aminopiperidine (120.0 g, 0.599 mol) was dissolved in 2-methyltetrahydrofuran (540 ml). Pyridine (58.14 ml, 0.719 mol) was added, followed by a line-wash of 2-methyltetrahydrofuran (60 ml). Chloroacetyl chloride (55.32 ml, 0.689 mol) was added dropwise, maintaining the temperature at about 21-25° C., followed by a line wash of 2-methyltetrahydrofuran (60 ml). After 2.5 h at ambient temperature, the reaction mixture was sampled for conversion to 6 by HPLC before the addition of a 16% w/w aqueous solution of sodium chloride (360 ml). The mixture was stirred for 30 min before separating off the aqueous phase.

To the organic phase was added a filtered solution of potassium thioacetate (102.65 g, 0.899 mol) in water (204 ml), followed by a line-wash of water (36 ml), maintaining the temperature at about 19-26° C. throughout. After stirring overnight at ambient temperature, the organic phase was sampled for conversion to 7 by HPLC before separating off the aqueous phase.

To the organic phase was added 4 (97.93 g, 0.539 mol) before dropwise addition of a solution of sodium methoxide in methanol (202 ml @ 25% w/w, 0.899 mol), maintaining the temperature at about 21-24° C. This was followed by a line wash of methanol (36 ml). After stirring for 1 h 50 min at ambient temperature, the reaction mixture was sampled by HPLC for conversion to 9 before heating to about 33° C., followed by dropwise addition of water (600 ml). After stirring for 10 min, the aqueous phase was separated off.

To the organic phase was added isohexane (960 ml) dropwise before removing a small sample of the reaction mixture, allowing it to cool and returning it to the bulk mixture to seed crystallisation. Dropwise addition of a second portion of isohexane (480 ml), followed by a ramped cool to about 3° C. over 1 h and a subsequent hold at this temperature overnight caused crystallisation of the product. Filtration, displacement washing the solid with ice-cold tert-butyl acetate (240 ml) and 2×ice-cold mixed solvent system of tert-butyl acetate and isohexane (1:1, 2×240 ml) and drying at about 40° C. over 3 days afforded 9 as a pale yellow solid (192.69 g, 77% yield based on 1-Boc-3-(S)-aminopiperidine).

1H NMR (400 MHz, DMSO-d6, 80° C.) δ: 7.49-7.32 (m, 3H), 7.19-7.12 (m, 1H), 7.01 (s, 1H), 6.91 (d, 1H), 6.29 (br, s, 1H), 3.91-3.64 (m, 3H), 2.96-2.77 (m, 2H), 1.92-1.77 (m, 1H), 1.74-1.30 (m, 12H).

Mass Spectrum: 420 [MH]⁺ and 364 [M-tBu]⁺.

### Example 3

### Synthesis of tert-butyl (3S)-3-({[5-(3-fluorophenyl)-3-{[(trichloroacetyl)carbamoyl]amino}thiophen-2-yl]carbonyl}amino)piperidine-1-carboxylate from compound 9 and trichloroacetyl isocyanate

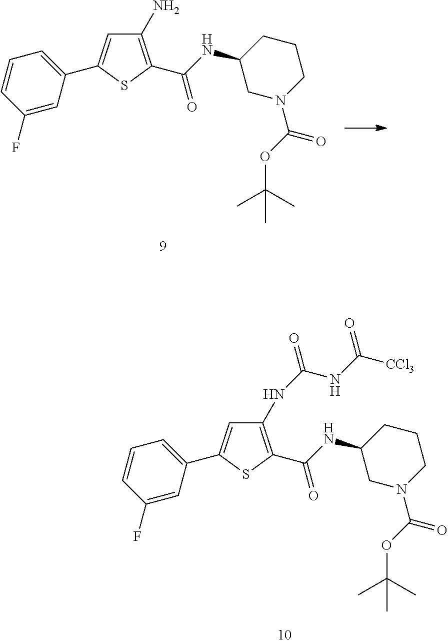

To a solution of 9 (73.12 g, 0.174 mol) in tetrahydrofuran (800 ml) was added trichloroacetyl isocyanate (23.23 ml, 0.196 mol), maintaining the temperature at about 20-30° C. during the addition. After 2.5 h at ambient temperature, the mixture was sampled for conversion to 10 before addition of isohexane (1120 ml) dropwise over 1 hour. After stirring for a further 1 h, the reaction mixture was filtered, the solid washed with isohexane (160 ml) and dried at about 40° C. to afford 10 as a pale peach solid (103.54 g, 98% yield).

1H NMR (400 MHz, DMSO-d6, 70° C.) δ: 11.70 (s, 1H), 11.49 (br. s, 1H), 8.24 (s, 1H), 7.80 (d, 1H), 7.57-7.40 (m, 3H), 7.26-7.18 (m, 1H), 3.97-3.67 (m, 3H), 2.95-2.78 (m, 2H), 1.97-1.84 (m, 1H), 1.78-1.53 (m, 2H), 1.51-1.33 (m, 10H).

13C NMR (400 MHz, DMSO-d6) δ: 162.3 (d, J=245 Hz), 161.7, 160.3, 153.7, 148.5, 141.9 (d, J=3 Hz), 140.5, 134.6 (d, J=8 Hz), 131.1 (d, J=9), 121.4 (d, J=3 Hz), 119.5, 115.3 (d, J=21 Hz), 114.7, 112.0 (d, J=23 Hz), 91.8, 78.4, 47.4, 45.7, 43.2, 29.2, 27.7, 23.2.

### Example 4

### Synthesis of tent-butyl (3S)-3-({[3-(ureido)-5-(3-fluorophenyl)thiophen-2-yl]carbonyl}amino)piperidine-1-carboxylate via deprotection of compound 10

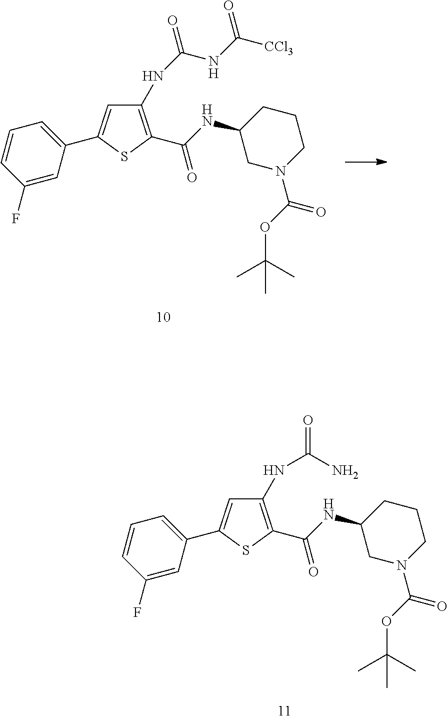

To a suspension of 10 (101.45 g, 0.169 mol) in methanol (516 ml) was added triethylamine (58.15 ml, 0.417 mol). After a further 2.5 h at ambient temperature, the mixture was sampled for conversion to 11 before addition of water (206 ml) over 10 min. After stirring overnight at ambient temperature, the reaction mixture was heated to about 45° C. for 15 min before addition of a second portion of water (1083 ml) over 2 h. After a further 1 h at about 45° C., the reaction mixture was allowed to cool to about 20° C. and held at this temperature for 1 h. The reaction mixture was filtered and the solid washed with water (206 ml) before drying at about 40° C. overnight to afford 10 as a white solid (77.10 g, 99% yield).

1H NMR (400 MHz, DMSO-d6, 80° C.) δ: 9.86 (s, 1H), 8.24 (s, 1H), 7.60-7.41 (m, 3H), 7.41-7.33 (m, 1H), 7.22-7.15 (m, 1H), 6.36 (br, s, 2H), 3.94-3.68 (m, 3H), 2.97-2.79 (m, 2H), 1.94-1.84 (m, 1H), 1.76-1.55 (m, 2H), 1.47-1.34 (m, 10H)

Mass Spectrum: 486 [MNa]⁺.

### Example 5

### Synthesis of 5-(3-Fluorophenyl)-3-ureidothiophene-2-carboxylic acid (S)-piperidin-3-ylamide via deprotection of compound II

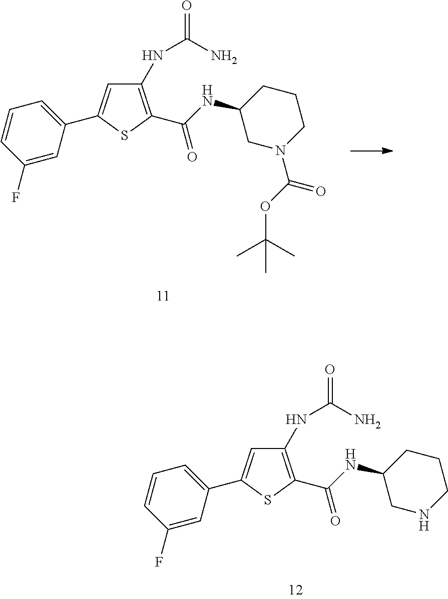

To a suspension of 11 (75.3 g, 0.163 mol) in methanol (383 ml) was added an aqueous solution of hydrochloric acid (40.78 ml @ 37% w/w in water, 0.488 mol) dropwise, maintaining the temperature at about 20-30° C. The resulting reaction mixture was then heated at about 50° C. for 4 h before sampling for conversion to 12. Triethylamine (85.10 ml, 0.610 mol) was added dropwise before addition of water (345 ml). A small sample of the reaction mixture was then removed, allowing it to cool before returning to the bulk mixture to seed crystallisation with stirring for 30 min. Further water (613 ml) was added over 1.5 h before holding at about 50° C. for a further 30 min and allowing to cool to about 20° C. with stirring overnight. The reaction mixture was filtered and the solid washed with water (153 ml) before drying at about 40° C. overnight to afford 12 as a white solid (57.26 g, 97% yield).

1H NMR (400 MHz, DMSO-d6, 80° C.) δ: 9.88 (br. s, 1H), 8.22 (s, 1H), 7.52-7.36 (m, 4H), 7.19 (m, 1H), 6.35 (br. s, 2H), 3.81 (m, 1H), 2.95 (m, 1H), 2.76 (m, 1H), 2.44-2.56 (m, 2H), 1.82 (m, 1H), 1.67-1.34 (m, 3H).

Mass Spectrum: 363 [MH]⁺.

### Example 6

### Purification of 5-(3-Fluorophenyl)-3-ureidothiophene-2-carboxylic acid (S)-piperidin-3-ylamide (compound 12)

A suspension of 12 (50.0 g, 0.138 mol) in methanol (650 ml) was heated to about 30° C. for 30 min before filtering the resulting hazy suspension through a 1.6 micron glass microfibre filter paper into a second vessel, followed by a line-wash with methanol (100 ml), discarding the solid residue. The resulting solution was cooled to about 10° C. before addition of water (250 ml), dropwise over 20 min, maintaining the temperature at about 10-15° C. To seed crystallisation, a sample of purified 12 was then added (150 mg, 0.3% wt/wt), and the contents of the vessel allowed to stir at about 10° C. for 30 min. Addition of a second portion of water (500 ml) over 1 h 30 min, maintaining the temperature at about 10-13° C., followed by stirring for 20 h at about 10° C., resulted in complete crystallisation. Filtration, washing the solid with water (2×100 ml), sucking dry for 30 min before drying under vacuum at about 40° C. overnight, afforded purified 12 as a white solid (46.91 g, 92% yield).

1H NMR (400 MHz, DMSO-d6) δ: 10.04 (s, 1H), 8.29 (s, 1H), 7.77 (d, 1H), 7.55-7.42 (m, 3H), 7.24 (m, 1H), 6.67 (br. s, 2H), 3.79 (m, 1H), 2.94 (m, 1H), 2.78 (m, 1H), 2.49-2.37 (m, 2H), 1.82 (m, 1H), 1.65-1.34 (m, 3H).

Mass Spectrum: 363 [MH]⁺.

### Example 7

### Synthesis of 5-(3-Fluorophenyl)-3-ureidothiophene-2-carboxylic acid (S)-piperidin-3-ylamide fumarate salt (compound 12 Fumarate salt)

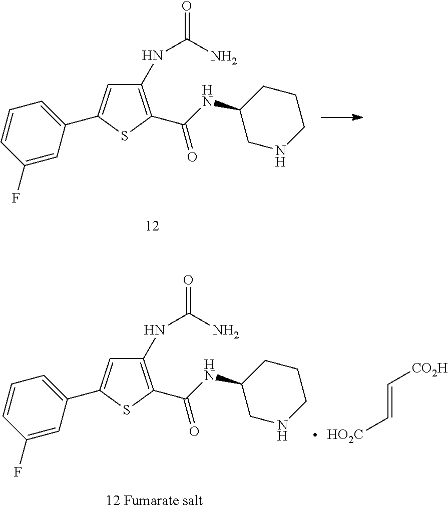

To a mixture of 12 (1.00 g, 2.8 mmol) and fumaric acid (160 mg, 1.4 mmol) was added acetone (3.0 ml) and water (1.9 ml). The resulting hazy solution was filtered through a syringe filter, before adding it dropwise to a second vessel containing a solution of fumaric acid (160 mg, 1.4 mmol) in acetone (18.5 ml) and water (0.5 ml), and a seed crystal of 12 Fumarate salt. The solution addition took place at ambient temperature over 1 h and was followed by a line-wash with acetone (1.0 ml) and water (0.1 ml). Gradual crystallisation of the product occurred, and after stirring the resulting slurry at ambient temperature for 1 h 30 min, the solid was filtered and washed with acetone (2×2.0 ml), sucking dry for 30 min before drying under vacuum at about 40° C. overnight to afford 12 Fumarate salt as a white solid (0.96 g, 96% yield).

1H NMR (400 MHz, DMSO-d6) δ: 10.00 (s, 1H), 8.29 (s, 1H), 8.24 (d, 1H), 7.54-7.42 (m, 3H), 7.24 (m, 1H), 6.67 (br. s, 2H), 6.52 (s, 2H [2H Fumaric acid]), 4.16 (br. m, 1H), 3.22 (m, 1H), 3.09 (m, 1H), 2.91-2.76 (m, 2H), 1.86 (m, 2H), 1.65 (m, 2H).

Mass Spectrum: 363 [MH]⁺.

### Example 8

### Synthesis of 5-(3-Fluorophenyl)-3-ureidothiophene-2-carboxylic acid (S)-piperidin-3-ylamide fumarate salt (compound 12 Hemi-Fumarate salt)

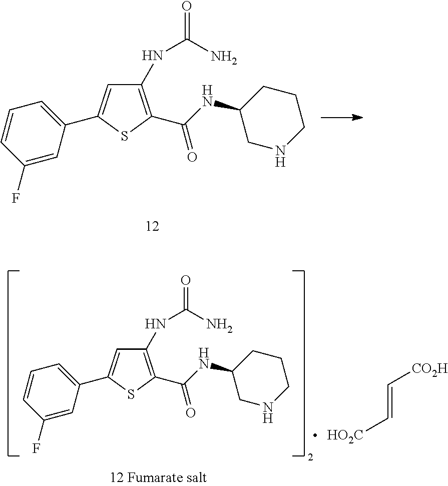

To a solution of 12 (2.0 g, 5.6 mmol) in methanol (33.7 ml) was added fumaric acid (327 mg, 2.8 mmol) and the resulting solution was stirred for 30 min at about 18° C. After seeding the solution with 12 Hemi-Fumarate salt (5 mg, 0.006 mmol) and stirring for 5 h at about 18-19° C., the reaction mixture was cooled to about 5° C., stirring was ceased and the reaction was held at this temperature overnight. Filtration of the resulting solid, washing with methanol (1×2 ml) and sucking dry on the filter afforded 12 Hemi-Fumarate salt as a white solid (1.90 g, 80%).

1H NMR (400 MHz, DMSO-d6) δ: 10.02 (s, 1H), 8.28 (s, 1H), 8.08 (d, 1H), 7.54-7.42 (m, 3H), 7.24 (m, 1H), 6.66 (br s., 2H), 6.47 (s, 1H [2H Fumaric acid]), 4.02 (br. m, 1H), 3.11 (m, 1H), 2.96 (m, 1H), 2.75-2.60 (m, 2H), 1.85 (m, 1H), 1.76 (m, 1H), 1.58 (m, 2H).

Mass Spectrum: 363 [MH]⁺.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for preparing a compound of formula I: 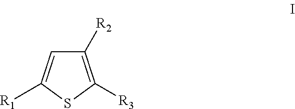
or a pharmaceutically acceptable salt thereof,
wherein:
R₁ is an aryl ring optionally substituted with one or more R₄ groups selected from halogen, C₁₋₆alkoxy, C₁₋₆alkoxycarbonyl, C₁₋₆alkyl, C₂₋₆alkenyl, C₂₋₆alkynyl, amido, amino, aryl, aryloxy, carboxy, cycloalkyl, heterocyclyl, and hydroxy;
R₂ is —NHC(O)NHR₅, where R₅ is selected from H, C₁₋₆alkyl, C₁₋₆alkoxycarbonyl, aryl, cycloalkyl, and heterocyclyl;
R₃ is —C(O)NR₆R₇, where R₆ and R₇ are each independently selected from H, C₁₋₆alkyl, cycloalkyl and a 5, 6, or 7-membered heterocyclyl ring containing at least one nitrogen atom, provided R₆ and R₇ are not both H;
comprising:
(a) reacting a 2-thioacetamide compound with a compound of formula II: 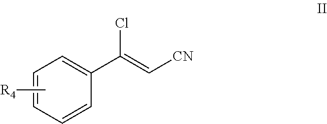
to produce an intermediate; and
(b) further reacting the intermediate to form the compound of formula I.

2. The method for preparing a compound of formula I according to claim 1 wherein the compound of formula I is: 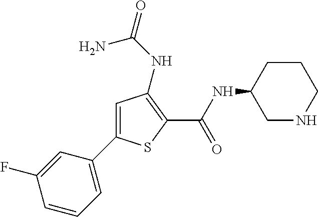
or a pharmaceutically acceptable salt thereof.

3. The method for preparing a compound of formula I according to claim 1 wherein the compound of formula II is:

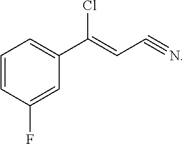

4. The method for preparing a compound of formula I according to claim 1, wherein reaction of the 2-thioacetamide compound with the compound of formula II takes place in the presence of a nucleophilic base.

5. The method for preparing a compound of formula I according to claim 1, wherein the 2-thioacetamide compound is formed in situ by deacetylating a precursor.

6. The method for preparing a compound of formula I according to claim 4 wherein the nucleophilic base is selected from sodium methoxide, sodium hydroxide, sodium or potassium ethoxide, sodium or potassium t-butoxide, and sodium t-amylate.

7. The method according to claim 2 wherein the pharmaceutically acceptable salt is a fumarate or hemi-fumarate salt.

8. A method for preparing a compound of formula I: 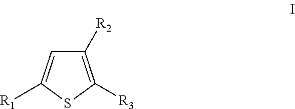
or a pharmaceutically acceptable salt thereof,
wherein:
R₁ is an aryl ring optionally substituted with one or more R₄ groups selected from halogen, C₁₋₆alkoxy, C₁₋₆alkoxycarbonyl, C₁₋₆alkyl, C₂₋₆alkenyl, C₂₋₆alkynyl, amido, amino, aryl, aryloxy, carboxy, cycloalkyl, heterocyclyl, and hydroxy;
R₂ is —NHC(O)NHR₅, where R₅ is selected from H, C₁₋₆alkyl, C₁₋₆alkoxycarbonyl, aryl, cycloalkyl, and heterocyclyl;
R₃ is —C(O)NR₆R₇, where R₆ and R₇ are each independently selected from H, C₁₋₆alkyl, cycloalkyl and a 5, 6, or 7-membered heterocyclyl ring containing at least one nitrogen atom, provided R₆ and R₇ are not both H;
comprising:
(a) reacting a thiophene intermediate of formula VII, or a pharmaceutically acceptable salt thereof 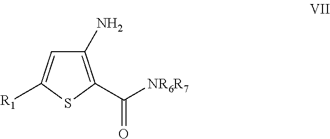
with an isocyanate to form a ureido intermediate;
(b) reacting the ureido intermediate with a base to form a urea intermediate; and
(c) further reacting the urea intermediate to form the compound of formula I.

9. The method for preparing a compound of formula I according to claim 8 wherein the compound of formula I is: 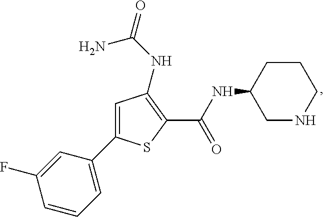
or a pharmaceutically acceptable salt thereof.

10. The method according to claim 9 wherein the pharmaceutically acceptable salt is a fumarate or hemi-fumarate salt.

11. A composition comprising a compound of formula I or a pharmaceutically acceptable salt thereof made by a process according to claim 1 and a pharmaceutically acceptable carrier.

12. A composition comprising a compound of formula I or a pharmaceutically acceptable salt thereof made by a process according to claim 8 and a pharmaceutically acceptable carrier.
